(19) United States
(12) Patent Application Publication
(10) Pub. No.: US 2023/0259698 A1
(43) Pub. Date: Aug. 17, 2023

(54) SYSTEM, METHOD AND PROGRAM FOR ASSISTING MEDICAL WORKERS

(71) Applicant: Medical Corporation Noukyuukai, Yokohama (JP)

(72) Inventors: Suiei Kaku, Yokohama (JP); Yuta Sugimoto, Yokohama (JP); Naoto Igarashi, Yokohama (JP); Takafumi Tomoi, Yokohama (JP)

(21) Appl. No.: 17/669,881

(22) Filed: Feb. 11, 2022

(51) Int. Cl. G06F 40/186; G06F 40/279; G10L 15/22; G10L 15/26; G16H 40/20

(52) U.S. Cl. CPC G06F 40/186; G06F 40/279; G10L 15/22; G10L 15/26; G16H 40/20

(57) ABSTRACT

A system is equipped with an audio data obtaining unit that obtains audio data of an explanation given to a patient by a physician, a data converting unit that converts the audio data obtained by the audio data obtaining unit into text data, a keyword extracting unit that extracts keywords which are set in advance from the text data converted by the data converting unit, a summary template storage unit that stores a plurality of summary templates in which explanations given to patients by physicians are summarized, and a summary template evaluating unit that evaluates the plurality of summary templates based on the keywords extracted by the keyword extracting unit.

### BACKGROUND OF THE INVENTION

### 1. Field of the Invention

The present disclosure is related to a system, method, and a program for assisting medical workers that records summaries of explanations given by physicians to patients in a simple manner.

### 2. Description of the Related Art

As in various other fields, work style reform is being promoted in the medical field recently. Long working hours and increases in the workloads of physicians are issues which are being pointed out. It is established that patients and their families have the right to listen to explanations of diseases and symptoms from physicians until they are satisfied. Meanwhile, among the responsibilities of physicians, providing such explanations consumes a great amount of time, and is a burdensome task.

Japanese Unexamined Patent Publication No. 2002-215804 proposes a method in which summary data of the medical states or diagnoses of patients generated by healthcare professionals other than physicians are cumulatively registered in an electronic healthcare/medical chart database.

### SUMMARY OF THE INVENTION

By registering summary data in a medical chart database as disclosed in Japanese Unexamined Patent Publication No. 2002-215804, the medical states or diagnoses of patients can be confirmed at a later point in time. Therefore, the burden of providing explanations to patients borne by physicians can be lessened, and transfer of responsibilities among physicians can also be facilitated.

However, if healthcare professionals other than physicians are required to generate the summary data as disclosed in Japanese Unexamined Patent Publication No. 2002-215804, the working hours of such healthcare professionals become longer and their workload increases. Therefore, this method is not a fundamental solution to the problem of increased workloads.

The present disclosure has been developed in view of the foregoing circumstances. The present disclosure provides a system, method, and program for assisting medical workers that can greatly reduce the workloads of physicians or healthcare professionals other than physicians.

The system for assisting medical workers of the present disclosure includes: an audio data obtaining unit that obtains audio data of an explanation given to a patient by a physician, a data converting unit that converts the audio data obtained by the audio data obtaining unit into text data, a keyword extracting unit that extracts keywords which are set in advance from the text data converted by the data converting unit, a summary template storage unit that stores a plurality of summary templates in which explanations given to patients by physicians are summarized, a summary template evaluating unit that evaluates the plurality of summary templates based on the keywords extracted by the keyword extracting unit, and a summary template recording unit that receives a selection of a summary template which is a target to be recorded from among the plurality of summary templates, and records the selected summary template.

According to the system for assisting medical workers of the present disclosure, audio data of an explanation given to a patient by a physician is obtained, the audio data is converted into text data, and keywords which are set in advance are extracted from the converted text data.

Next, the plurality of summary template data which are stored in advance are referred to, and the plurality of summary templates are evaluated based on the extracted keywords. Thereafter, selection of a summary template which is a target to be recorded from among the plurality of evaluated summary templates is received, and the selected summary template is recorded.

Thereby, it becomes possible for a physician or a healthcare professional other than a physician to generate a summary of the explanation of a disease efficiently, and the workload of the physician or the healthcare professional other than a physician can be greatly reduced.

In addition, explanations of diseases can be standardized and made transparent, resulting in the contents of explanations of diseases given by physicians becoming sharable among many professionals. Thereby, the number of times that nurses, etc. ask physicians to confirm the contents of explanations that they have given can be reduced.

### BRIEF DESCRIPTION OF THE DRAWINGS

FIG. 1 is a block diagram that illustrates the schematic configuration of an embodiment of a system for assisting medical workers of the present disclosure.

FIG. 2 is a diagram that illustrates an example of summary templates and keywords which are correlated with the summary template.

FIG. 3 is a diagram that illustrates an example of a user interface screen.

FIG. 4 is a diagram that illustrates an example of a summary template editing screen.

FIG. 5 is a flow chart for explaining the flow of processes of the entire system for assisting medical workers illustrated in FIG. 1.

### DETAILED DESCRIPTION OF THE INVENTION

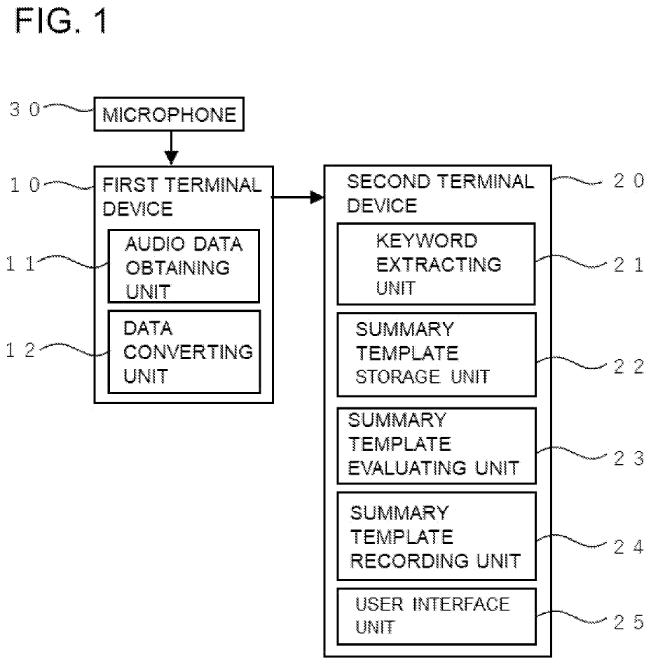

An embodiment of a system for assisting medical workers of the present disclosure will be described in detail with reference to the attached drawings. FIG. 1 is a block diagram that illustrates the schematic configuration of an embodiment of a system for assisting medical workers 1 of the present disclosure.

The system for assisting medical workers 1 of the present embodiment obtains audio data when a physician explains a disease or the like to a patient, and converts the audio data to text data. Then, the system for assisting medical workers 1 extracts keywords which are set in advance from the text data, and evaluates a plurality of summary templates which are recorded in advance based on the extracted keywords. A physician or a healthcare professional other than a physician selects a summary template to be recorded by referring to the evaluation results for each of the summary templates. The system for assisting medical workers 1 records the summary template which is selected by the physician or the healthcare professional other than a physician in an electronic medical chart, etc. of the patient.

Specifically, the system for assisting medical workers 1 is equipped with a first terminal device 10 and a second terminal device 20, as illustrated in FIG. 1.

Each of the first terminal device 10 and the second terminal device 20 is constituted by a computer, and is equipped with a CPU (Central Processing Unit), a semiconductor memory, a hard disk, a communications I/F (Interface), etc. The first terminal device 10 and the second terminal device 20 are constituted by desktop personal computers, laptop computers, tablet terminals, etc. The second terminal device 20 displays a user interface screen and receives input of various settings on the user interface screen as will be described later, and therefore it is preferable for the second terminal device 20 to be a tablet terminal having a touch panel.

The first terminal device 10 and the second terminal device 20 are connected via the Internet or a LAN (Local Area Network), and are configured to be capable of communicating with each other.

In addition, the first terminal device 10 is connected to a microphone 30 for recording explanations given to patients by physicians, as illustrated in FIG. 1. It is preferable for the microphone 30 to be a lapel microphone which can be attached to the chest of a physician, for example.

The first terminal device 10 is installed in a room in which a physician explains diseases and treatments to a patient, for example, and is equipped with an audio data obtaining unit 11 and a data converting unit 12 as illustrated in FIG. 1.

The audio data obtaining unit 11 obtains audio data which is collected and amplified by the microphone 30.

The data converting unit 12 converts the audio data obtained by the audio data obtaining unit 11 into text data. With respect to the conversion from audio data to text data, known applications, systems, or the like may be employed.

The first terminal device 10 converts the text data which was converted by the data converting unit 12 into a PDF file, and outputs the PDF file to the second terminal device 20. Known applications such as Air Drop™ may be employed as the method of outputting the PDF file from the first terminal device 10 to the second terminal device 20. Alternatively, the PDF file may be sent by e-mail, or delivered to the second terminal device 20 via a predetermined data server apparatus (not shown).

The second terminal device 20 is equipped with a keyword extracting unit 21, a summary template storage unit 22, a summary template evaluating unit 23, a summary template recording unit 24, and a user interface unit 25 as illustrated in FIG. 1. Note that in the present embodiment, the user interface unit 25 corresponds to an editing command receiving unit and a signature input receiving unit of the present disclosure.

An embodiment of a program for assisting medical workers of the present disclosure is installed in the hard disk or the semiconductor memory of the second terminal device 20. The keyword extracting unit 21, the summary template evaluating unit 23, the summary template recording unit 24, and the user interface unit 25 function by the program for assisting medical workers being executed by the CPU of the second terminal device 20.

Note that in the present embodiment, the functions of the keyword extracting unit 21, the summary template evaluating unit 23, the summary template recording unit 24 and the user interface unit 25 are realized by software as described above. However, the present disclosure is not limited to such a configuration, and a portion or the entirety of the aforementioned functions may be executed by hardware components such as an ASIC (Application Specific Integrated Circuit), an FPGA (Field Programmable Gate Army), or other electric circuits.

In addition, the summary template storage unit 22 is constituted by the hard disk or the semiconductor memory of the second terminal device 20, and stores summary templates. The summary templates are summaries of explanations regarding diseases and treatment plans given to patients by physicians, and are constituted by words, phrases, and sentences which are determined in advance.

In the present embodiment, the summary templates are produced for each combination of the type of disease and the timing at which a patient is given an explanation by a physician, and stored in the summary template storage unit 22. The contents of explanations regarding diseases given by physicians follow certain patterns for each disease or for timings at which the physicians give explanations. Therefore, the present embodiment produces templates of such summaries in advance and stores them in the summary template storage unit 22.

Examples of diseases include “cerebral infarction”, “vertigo”, “intracerebral hemorrhage”, “subarachnoidal hemorrhage”, “chronic subdural hematoma”, etc. The timings at which patients are given explanations by physicians include “Admission to Hospital”, “Interim Report”, “Discharge from Hospital”, etc. Note that the diseases and the timings at which patients are given explanations by physicians are not limited to the above, and other diseases and timings may be set as well.

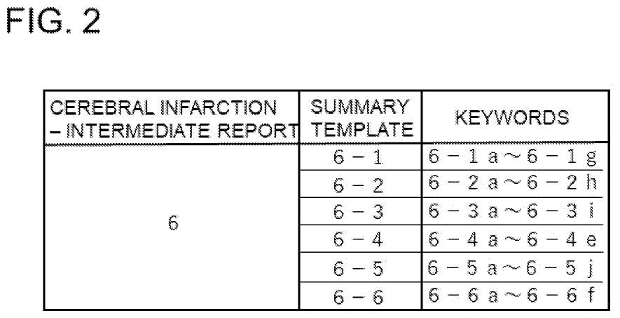

FIG. 2 is a diagram that illustrates an example of summary templates for a case in which the disease is “cerebral infarction” and the timing at which a physician gives an explanation is “Interim Report”. As illustrated in FIG. 2, the number “6” is designated for a combination of“cerebral infarction” and “Interim Report”, and six types of summary templates 6-1 through 6-6 that correspond to the number “6” are stored in the summary template storage unit 22.

Note that FIG. 2 illustrates an example of summary templates for a case in which the disease is “cerebral infarction” and the timing at which a physician gives an explanation is “Interim Report”. In actuality, a number is designated for each combination of diseases and timings at which a physician gives an explanation, and a plurality of summary templates corresponding to such numbers are stored in the summary template storage unit 22. For example, the number “5” is designated for a combination of“cerebral infarction” and “Admission to Hospital” and six types of summary templates 5-1 through 5-6 are stored, and the number “4” is designated for a combination of “intracerebral hemorrhage” and “Discharge from Hospital”, and six types of summary templates 4-1 through 4-6 are stored.

The keyword extracting unit 21 obtains text data from the PDF data which was output from the first terminal device 10, and extracts keywords which are set in advance from the text data. As a method for extracting the text data from the PDF data, a user of the second terminal device 20 may copy and paste the text data which is included in the PDF data to obtain the text data, for example. Alternatively, a known character recognition process ma be administered on the PDF data to obtain the text data.

The keywords which are set in advance are keywords related to the aforementioned summary templates. A plurality of keywords are set for each of the summary templates. In the example illustrated in FIG. 2, 10 keywords 6-5a through 6-5j are set with respect to a template “6-5”, which is one of the summary templates for a combination of “cerebral infarction” and “Interim Report”, for example. Examples of keywords include “new infarction”, “prevent deterioration”, “occluded”, “about 10 days”, and “physical therapy”. However, the keywords are not limited to these, and any words for which there is a possibility that they will be utilized in an Interim report to a patient with cerebral infarction may be included. In addition, a plurality of keywords are set with respect to each of the summary templates 6-1 through 6-4 and 6-6, in addition to the “6-5” summary template. Further, a plurality of keywords are set with respect to the summary templates designated by numbers other than the number “6”.

The keyword extracting unit 21 compares the text data output from the first terminal device 10 and all of the aforementioned keywords which are set in advance, and extracts the keywords which are included in the text data. Note that known techniques may be employed for the keyword extraction process.

The summary template evaluating unit 23 evaluates each of the plurality of summary templates which are stored in the summary template storage unit 22, based on the keywords which are extracted by the keyword extracting unit 21.

The summary template evaluating unit 23 specifies summary templates corresponding to a combination of a disease name and a timing at which a physician gave an explanation from among the plurality of summary templates, based on a combination of a disease name and a timing at which a physician gave an explanation that were input as settings by healthcare professionals other than a physician, etc. Each of the specified summary templates are evaluated based on how many keywords corresponding thereto are included in the text data.

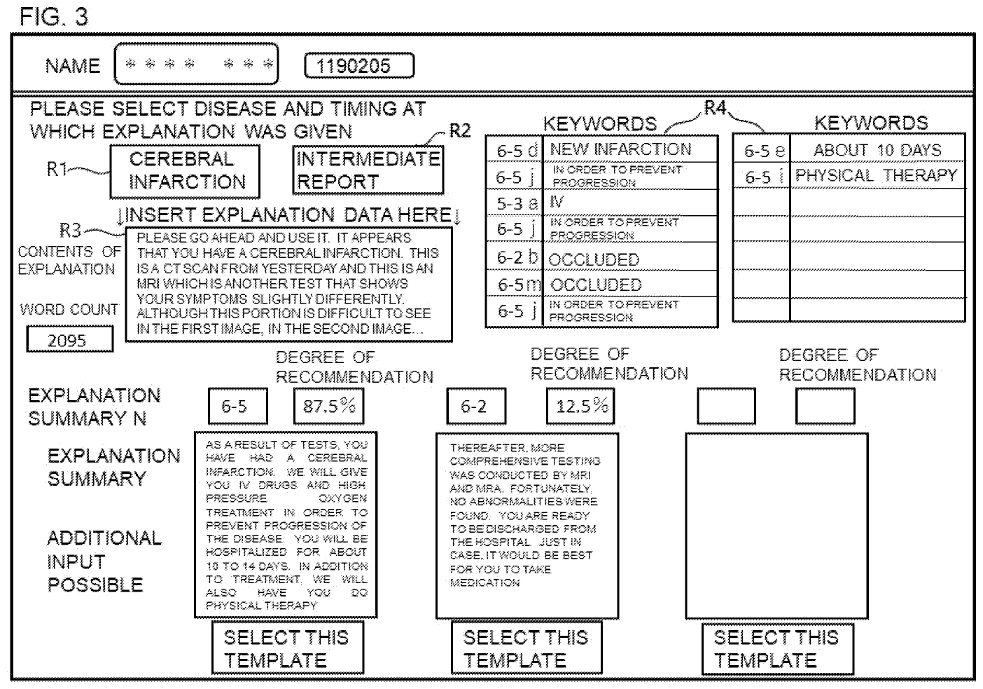

Here, the method by which the summary template evaluating unit 23 of the present embodiment evaluates the summary templates will be described with reference to the user interface screen illustrated in FIG. 3. The user interface screen illustrated in FIG. 3 is a screen which is displayed on a display of the second terminal device 20 by the user interface unit 25.

First, the user interface screen which is displayed by the second terminal device 20 will be described. The name and the identification number of a patient is displayed at the upper left portion of the user interface screen, and an entry field R1 for a disease name and an entry field R2 for a timing at which an explanation was given to the patient are displayed thereunder. The disease name entry field R1 and the explanation timing entry field R2 are both pull down menus. The disease name and the timing of the explanation are selected from the pull down menu. Note that the present disclosure is not limited to utilizing pull down menus, and a user may input text into the entry field R1 and the entry field R2.

An entry field R3 into which the text data of the explanation given to the patient by a physician is displayed under the entry fields R1 and R2. The user may input the text data into the entry field R3 by copying and pasting, for example. The word count of the text data is displayed toward the left of the entry field R3.

In the case that the text data of the physician's explanation is input into the entry field R3, the keywords which are set in advance are extracted therefrom by the keyword extracting unit 21 as described above. Then, the keywords which are extracted by the keyword extracting unit 21 are displayed in a keyword display field R4 toward the right of the entry field R3. FIG. 3 illustrates an example in which keywords corresponding to summary template “6-5”, keywords corresponding to summary template “6-2”, and keywords corresponding to summary template “5-3” are extracted.

Summary templates and evaluation values thereof are displayed in the lower half of the user interface screen.

Specifically, the summary template evaluating unit 23 specifies summary templates corresponding to a combination of the disease which is input into the entry field R1 and the timing at which a physician gave an explanation which is input into the entry field R2, based on the combination which is input, from among the plurality of summary templates. For example, in the case that the disease is “cerebral infarction” and the timing at which the physician gave the explanation is “Interim Report”, the summary templates 6-1 through 6-6 of number “6” illustrated in FIG. 2 are specified.

Next, the summary template evaluating unit 23 compares the keywords which are set in advance as corresponding to the specified summary templates and the keywords which are extracted by the keyword extracting unit 21, to specify summary templates which are to be targets of evaluation.

Specifically, the summary template evaluating unit 23 specifies summary templates that correspond to the keywords which are extracted by the keyword extracting unit 21, from among the summary templates 6-1 through 6-6. In the case of the example illustrated in FIG. 3, keywords corresponding to summary template 6-5 (6-5d, 6-Se, 6-5i, 6-5j, and 6-5m) and keywords corresponding to summary template 6-2 (6-2b) are extracted by the keyword extracting unit 21. Therefore, the summary template 6-5 and the summary template 6-2 are specified as targets of evaluation. Note that in the case of the example illustrated in FIG. 3, the keyword 5-3a “IV drip” is also extracted. However, summary template 5-3 is not a summary template that corresponds to a combination of “cerebral infarction” and “Interim report”, and therefore the summary template evaluating unit 23 does not specify the summary template 5-3 as a target of evaluation.

Next, the summary template evaluating unit 23 evaluates the summary templates 6-5 and 6-2 which have been specified as targets for evaluation. Specifically, in the case of the example illustrated in FIG. 3, the number of keywords which are extracted by the keyword extracting unit 21 that correspond to the combination of “cerebral infarction” and “Interim Report” is eight. From among the eight, the number of keywords that correspond to the summary template 6-5 is seven, and the number of keywords that correspond to the summary template 6-2 is one.

In this case, the summary template evaluating unit 23 calculates ⅞·100=87.5% as an evaluation value for the summary template 6-5, and calculates ⅛·100=12.5% as an evaluation value for the summary template 6-2.

Then, the evaluation results which are calculated by the summary template evaluating unit 23 are displayed in the lower half portion of the user interface screen by the user interface unit 25. Specifically, in the case of the example illustrated in FIG. 3, the user interface unit 25 displays the summary template 6-5 having the highest evaluation value at the leftmost portion, and displays the summary template 6-2 having the next highest evaluation value to the right of the summary template 6-5. That is, the user interface unit 25 displays the summary template having the highest evaluation value at the leftmost portion, and displays summary templates from the left to the right in descending order of evaluation values. Thereby, a physician or a healthcare professional other than a physician can instantly understand the order of the evaluation values of the plurality of summary templates.

In addition, the user interface unit 25 displays the evaluation values of each of the summary templates which are targets of evaluation above the displayed summary templates as degrees of recommendation. Then, a physician selects a summary template to be ultimately recorded by referring to the evaluation values which are displayed as degrees of recommendation. Note that the summary template which is selected by a physician need not necessarily be that having the highest evaluation value, and a physician may select a summary template having a lower evaluation value in cases that such a summary template is more appropriate for being closer to the contents which were explained to a patient.

Selection of the summary template which is the target to be recorded is conducted by pressing the button “Select This Template” displayed under each of the summary templates which are targets of evaluation illustrated in FIG. 3.

The summary template recording unit 24 receives the selection of the summary template which is determined by the “Select This Template” button being pressed, and records the selected summary template. The recording destination may be the hard disk of the second terminal device 20, or may be a separate server apparatus which is connected to the second terminal device 20 via a communications circuit.

Summary templates are recorded for each patient and for each timing that explanations are given (during a hospital stay, an interim report, etc.), such that they can be read out as necessary.

Note that when recording the summary templates, the summary templates may be recorded as they are stored in advance. Alternatively, a physician may edit a summary template by adding to the summary template after confirming the contents thereof.

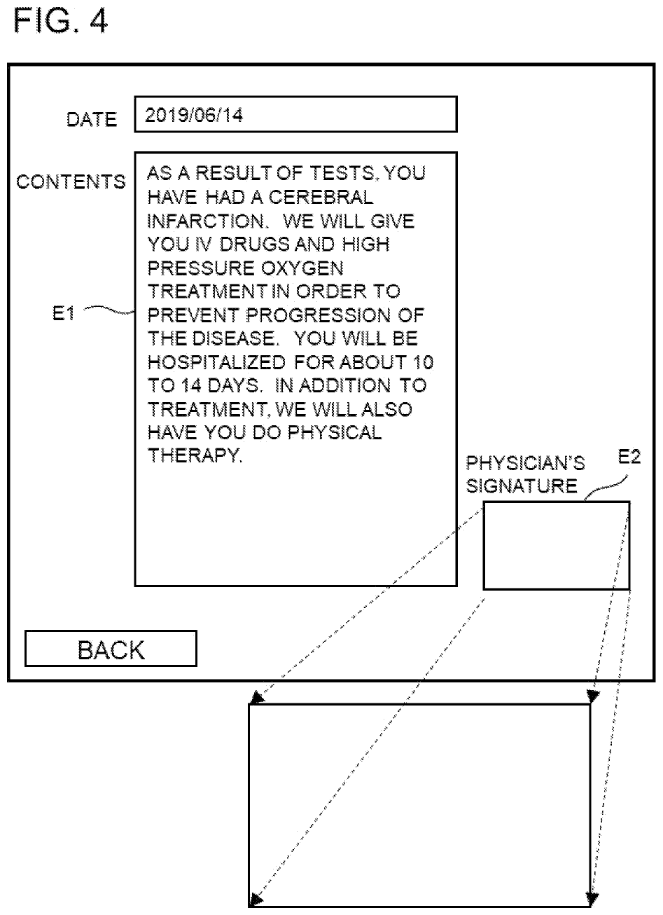

Specifically, after pressing the “Select This Template” as described above, the user interface unit 25 causes a summary template editing screen such as that illustrated in FIG. 4 on the second terminal device 20. The contents of the summary template are displayed in an editing field E1 on the summary template editing screen. A physician can confirm the contents which are displayed in the editing field E1 and add or delete text to complete a desired summary. With respect to the editing operations, a keyboard capable of inputting text may be displayed on the summary template editing screen to receive editing operations, or a separate input device which is connected to the second terminal device 20 may be employed to receive the editing operations. By enabling editing operations on the summary template, a more appropriate summary can be recorded.

Further, the user interface unit 25 displays a signature field E2 at the lower right of the editing field E1. The signature field E2 is a field for receiving input of a physician's signature. A physician confirms the contents of the edited summary, and signs the signature field E2 if there are no issues. With respect to the signature in the signature field E2, the signature field E2 may be a signature may be enlarged and displayed as illustrated by the arrow in dotted lines in FIG. 4, and may be input by a stylus pen or by a finger.

The summary template recording unit 24 records the edited summary at a point in time when input of a signature by a physician is received. Note that the summary template recording unit 24 is not limited to recording a summary template in cases that the summary template is edited, but also may record a summary template as is, after input of the signature of a physician is received.

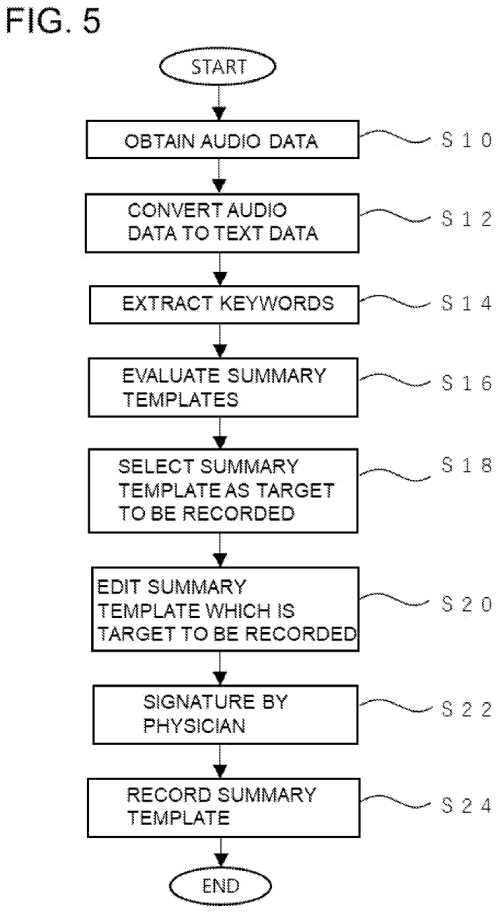

Next, the entirety of the flow of processes conducted by the system for assisting medical workers 1 of the present embodiment will be described with reference to the flow chart illustrated in FIG. 5.

First, an explanation regarding a disease, a treatment plan, etc. is given to a patient by a physician, and audio data of the contents of the explanation is obtained by the first terminal device 10 via the microphone 30 (S10).

Next, the audio data obtained by the first terminal device 10 is converted into text data, and is output to the second terminal device 20 as a PDF file (S12).

The second terminal device 20 obtains text data from the PDF file input thereto, and extracts keywords which are set in advance from the text data (S14). Specifically, the keyword extracting unit 21 of the second terminal device 20 compares the text data and keywords correlated to summary templates in advance, and extracts keywords which are included in the text data.

Then, the summary template evaluating unit 23 specifies summary templates which are to be targets of evaluation based on the keywords which are extracted by the keyword extracting unit 21, and evaluates the specified summary templates (S16).

Thereafter, the evaluation results calculated by the summary template evaluating unit 23 are displayed along with the summary templates as “degrees of recommendation”, and a physician or healthcare professionals other than a physician selects a summary template to be ultimately recorded by referring to the evaluation values as degrees of recommendation (S18).

The summary template selected by the physician or the healthcare professional other than a physician is displayed on the summary template editing screen, and editing operations are conducted by a physician as necessary (S20).

After the editing operations on the summary template are completed, a signature is input into the signature field E2 by the physician (S22). Thereafter, the summary template recording unit 24 records the edited summary template in the second terminal device 20 (S24).

Note that in the system for assisting medical workers of the above embodiment, the audio data obtaining unit 11 and the data converting unit 12 are provided in the first terminal device 10, and the keyword extracting unit 21, the summary template storage unit 22, the summary template evaluating unit 23, the summary template recording unit 24, and the user interface unit 25 are provided in the second terminal device 20. However, the present disclosure is not limited to such a configuration, and the audio data obtaining unit 11, the data converting unit 12. the keyword extracting unit 21, the summary template storage unit 22, the summary template evaluating unit 23, the summary template recording unit 24, and the user interface unit 25 may be provided in a single terminal device.

The additional items are disclosed in relation with the system for assisting medical workers of the present disclosure.

### (Additional Items)

In the system for assisting medical workers of the present disclosure, the keywords may be keywords which are related to the contents of the summary templates.

In addition, in the system for assisting medical workers of the present disclosure, the summary template storage unit may store a plurality of summary templates for each combination of a disease and a timing at which a patient is given an explanation.

Further, the system for assisting medical workers of the present disclosure may also be equipped with an editing command receiving unit that receives editing commands with respect to a summary template which is selected as a target to be recorded.

Still further, the system for assisting medical workers of the present disclosure may also be equipped with a signature input receiving unit that receives input of the signature of a physician when a summary template which is selected as a target to be recorded is recorded.

A method for assisting medical workers of the present disclosure stores a plurality of summary templates in which explanations given to patients by physicians are summarized in advance, obtains audio data of an explanation given to a patient by a physician, converts the audio data to text data, extracts keywords which are set in advance from the converted text data, evaluates the plurality of summary templates based on the extracted keywords, receives selection of a summary template as a target to be recorded from among the plurality of summary templates, and records the selected summary template.

A recording medium on which a program for assisting medical workers of the present disclosure is recorded causes a computer to execute a step of extracting keywords which are set in advance from text data obtained by converting audio data of an explanation given to a patient by a physician into text, a step of referring to a plurality of templates that summarize explanations given to patients by physicians which are stored in advance and evaluating the plurality of summary templates based on the extracted keywords, and a step of receiving selection of a summary template as a target to be recorded from among the plurality of summary templates, and recording the selected summary template.

What is claimed is:

1. A system for assisting medical workers comprising:
an audio data obtaining unit that obtains audio data of an explanation given to a patient by a physician;
a data converting unit that converts the audio data obtained by the audio data obtaining unit into text data;
a keyword extracting unit that extracts keywords which are set in advance from the text data converted by the data converting unit;
a summary template storage unit that stores a plurality of summary templates in which explanations given to patients by physicians are summarized; and
a summary template evaluating unit that evaluates the plurality of summary templates based on the keywords extracted by the keyword extracting unit.

2. The system for assisting medical workers according to claim 1, further comprising:
a summary template recording unit that receives a selection of a summary template which is a target to be recorded from among the plurality of summary templates, and records the selected summary template.

3. The system for assisting medical workers according to claim 1, wherein:
the keywords are keywords which are related to the contents of the summary templates.

4. The system for assisting medical workers according to claim 1, wherein:
the summary template storage unit stores a plurality of templates for each combination of diseases and timings at which patients are given explanations.

5. The system for assisting medical workers according to claim 1, further comprising:
an edit command receiving unit that receives commands to edit the summary template which is selected as a target to be recorded.

6. The system for assisting medical workers according to claim 1, further comprising:
a signature input receiving unit that receives input of the signature of the physician when recording the summary template which is selected as a target to be recorded.

7. A method for assisting medical workers, comprising:
storing a plurality of summary templates in which explanations given to patients by physicians are summarized in advance;
obtaining audio data of an explanation given to a patient by a physician;
converting the audio data to text data;
extracting keywords which are set in advance from the converted text data; and
evaluating the plurality of summary templates based on the extracted keywords.

8. A recording medium on which a program for assisting medical workers is recorded that causes a computer to execute:
a step of extracting keywords which are set in advance from text data obtained by converting audio data of an explanation given to a patient by a physician into text; and
a step of referring to a plurality of templates that summarize explanations given to patients by physicians which are stored in advance and evaluating the plurality of summary templates based on the extracted keywords.
